(19) United States
(12) Patent Application Publication
(10) Pub. No.: US 2023/0056975 A1
(43) Pub. Date: Feb. 23, 2023

(54) BED SHEET AND ASSOCIATED ASSEMBLY

(71) Applicant: Purifas Holdings Pty Ltd, Bankstown (AU)

(72) Inventor: Ibrahim Samaan, Bankstown (AU)

(21) Appl. No.: 17/893,816

(22) Filed: Aug. 23, 2022

(30) Foreign Application Priority Data
Aug. 23, 2021 (AU) 2021221404
Sep. 27, 2021 (AU) 2021107569

(51) Int. Cl. A47G 9/00; A47G 9/02

(52) U.S. Cl. CPC A47G 9/007; A47G 9/0238

(57) ABSTRACT

A bed sheet and securement assembly and a bed sheet and dispenser assembly include an elongate sheet which is one of a plurality of sheets provided in a dispensable roll of separable sheets. The bed sheet and securement assembly include the elongate sheet being adapted to cover an exposed or shared upper surface of a bed and including securement openings and formed adjacent to each of its opposing ends and securement means and adapted to mount to the bed or proximal its opposing ends and arranged for releasable engagement with the elongate sheet via the securement openings and for securement of the elongate sheet to the therapy bed.

### CROSS-REFERENCE TO RELATED APPLICATIONS

The present disclosure claims the benefit of and priority to Australian Patent Application No. 2021107569, filed on Sep. 27, 2021, which is a divisional of Australian Patent Application No. 2021221404, filed on Aug. 23, 2021, the entire content of each of which is hereby incorporated by reference herein.

### TECHNICAL FIELD

The present disclosure is broadly directed to a bed sheet and securement assembly. The disclosure is also generally directed to a bed sheet and dispenser assembly.

### BACKGROUND

In medical environments it is known to cover a patient's bed with a disposable bed sheet. The bed sheet is typically dispensed from a roll and, in a similar manner to hand towels, is torn off the roll at the required length. The disposable bed sheet is draped across exposed surfaces of the bed. Although relatively inexpensive, this style of disposable bed sheet is ineffective in (a) providing adequate coverage of the exposed bed surfaces, and (b) fitting to the bed without sliding relative to the bed and exposing previously covered surfaces. Another style of existing bed sheet intended to address these shortcomings of the low-cost disposable sheets are fitted bed sheets. Fitted bed sheets may be disposable or re-usable and incorporate elastic or other means around their perimeter to fit about the edge of the bed, akin to fitted bed sheets used on a mattress. The fitted bed sheets are non-recyclable and relatively expensive and as such it is generally not commercially viable to use disposable fitted sheets in a hospital, clinical or other high volume environment. For this reason the re-usable version of the fitted sheet may be preferred although laundering and associated costs are a disincentive for deploying re-usable fitted bed sheets.

### SUMMARY OF DISCLOSURE

Accordingly to a first aspect of the present disclosure there is provided a bed sheet and securement assembly comprising:

an elongate sheet being one of a plurality of sheets provided in a dispensable roll of separable sheets, said elongate sheet (i) being adapted to cover exposed or shared surfaces of a bed, and (ii) including securement openings formed adjacent to each of opposing ends of said sheet;

securement means adapted to mount to the bed at or proximal its opposing ends and arranged for releasable engagement with the elongate sheet via the securement openings for securement of the elongate sheet to the bed.

In embodiments, the securement means each include a hook fixture configured to cooperate with respective of the securement openings formed in the elongate sheet for releasable engagement of said openings with said hook fixtures for securement of said sheet to the bed. It is contemplated that each of the hook fixtures includes an elongate member connected to a base mounting of the securement for mounting to the bed, the securement openings being shaped at least in part complementary to the elongate member. In aspects, the elongate member includes a pair of prongs extending laterally either side of the base mounting and configured for releasable engagement of the elongate sheet about its securement openings.

The securement openings of the elongate sheet are each formed as an aperture in said sheet. In embodiments, the apertures are aligned with the elongate member of the hook members mounted at or proximal each end of the bed. It is contemplated that the apertures are longitudinally separated a predetermined distance for fitting of the elongate sheet to the exposed or shared surface of the bed via the hook fixtures mounted at opposing ends of said bed.

The elongate sheet is formed in one piece from a recyclable and non-woven material. In aspects, the recyclable material is formed in multiple layers being effective in bacterial filtration. In embodiments, the one-piece elongate sheet completely covers the exposed or shared surface of the bed.

According to a second aspect of the present disclosure there is provided a bed sheet and dispenser assembly comprising:

a dispensable roll of a plurality of separable elongate sheets each being adapted to cover an exposed or shared surface of a bed, said sheets each including (i) securement openings formed adjacent to each of opposing ends of said sheet and adapted for releasable engagement with securement means mounted to the bed for securement of said sheet to said bed, and (ii) a tear line defined by a plurality of adjoining tear junctions for adjacent of the separable sheets from the roll;

a sheet dispenser configured for housing and tearing of the dispensable roll, said dispenser including severing means arranged for alignment with the adjoining tear junctions of adjacent of the sheets for tearing of each of said sheets from the roll.

In aspects, the severing means includes a series of teeth arranged for engagement with the dispensable roll across one of the tear lines wherein the teeth locate between neighbouring of the adjoining tear junctions to facilitate tearing of each of said sheets from the roll. In embodiments, each of the adjoining tear junctions includes a plurality of frangible tear points located in close proximity to one another.

In other aspects, the sheet dispenser includes capture means located adjacent the severing means and configured for releasable engagement of one of the securement openings to restrict retraction of the associated sheet within the dispenser during dispensing of one of the sheets from the roll. In embodiments, the capture means includes a fixed latching member configured to cooperate with the securement opening to latch with said opening in the event the associated sheet retracts within the dispenser.

### BRIEF DESCRIPTION OF DRAWINGS

In order to achieve a better understanding of the nature of the present disclosure an embodiment of a bed sheet and securement assembly together with a bed sheet and dispenser assembly of alternative aspects of the disclosure will now be described, by way of example only, with reference to the accompanying drawings in which:

FIG. 1 is a perspective view of an embodiment of both a bed sheet and securement assembly as well as a bed sheet and dispenser assembly according to alternative aspects of the disclosure;

FIGS. 2 and 3 are perspective views of an embodiment of a bed sheet and anchorage assembly according to a first aspect of the disclosure;

FIG. 4 is a perspective view of an embodiment of a bed sheet and dispenser assembly according to a second aspect of the disclosure;

FIG. 5 is an enlarged detail view of the embodiment of the first aspect of the disclosure taken from FIG. 2;

FIG. 6 is an enlarged detail view of the embodiment of the second aspect of the disclosure taken from FIG. 4;

FIG. 7 is a perspective view of the embodiment of the second aspect of the disclosure showing a dispensable roll of separable elongate sheets removed from an associated sheet dispenser;

FIG. 8 is a plan view of one of the plurality of sheets taken from a dispensable roll of the embodiment of both aspects of the disclosure and being shown broken longitudinally to indicate its indefinite extent; and

FIG. 9 is an enlarged detail view of the embodiment of the elongate sheet of both aspects of the disclosure taken from FIG. 8.

### DETAILED DESCRIPTION

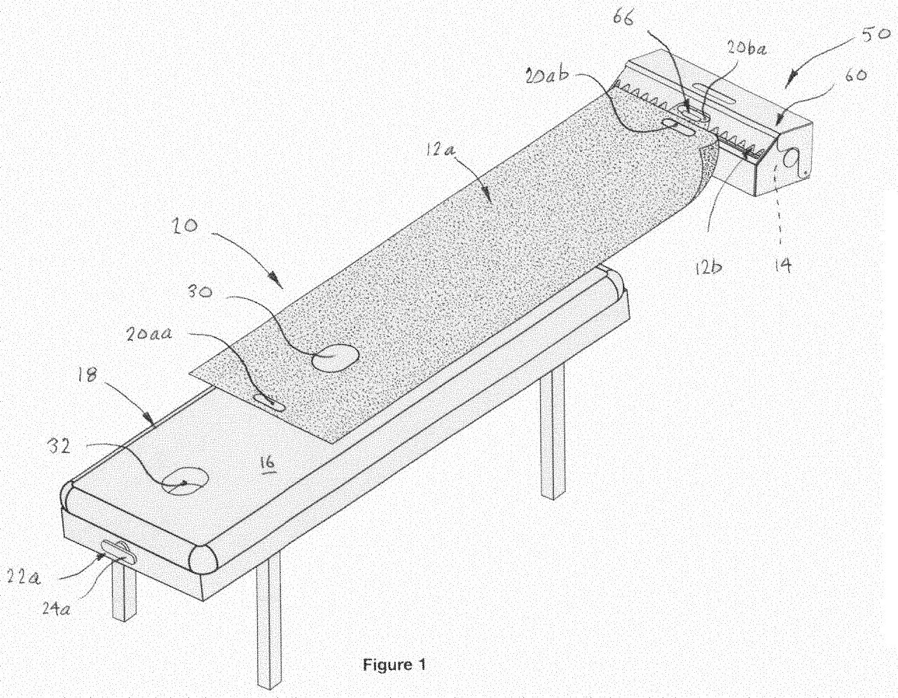

As seen in FIG. 1, there is an embodiment of both aspects of the disclosure which is in a first aspect broadly directed to a bed sheet and securement assembly 10 and in a second aspect a bed sheet and dispenser assembly 50. In both aspects the securement assembly 10 and the dispenser assembly 50 include an elongate sheet such as 12a which is one of a plurality of sheets such as 12a and 12b provided in a dispensable roll 14 of separable sheets 12a/b. In this embodiment each of the sheets 12a is adapted to cover an exposed or shared upper surface 16 of a bed such as a therapy bed 18. It is to be understood that the disclosure extends to other beds typically used in a medical environment including but not limited to beds at a medical practice or hospital.

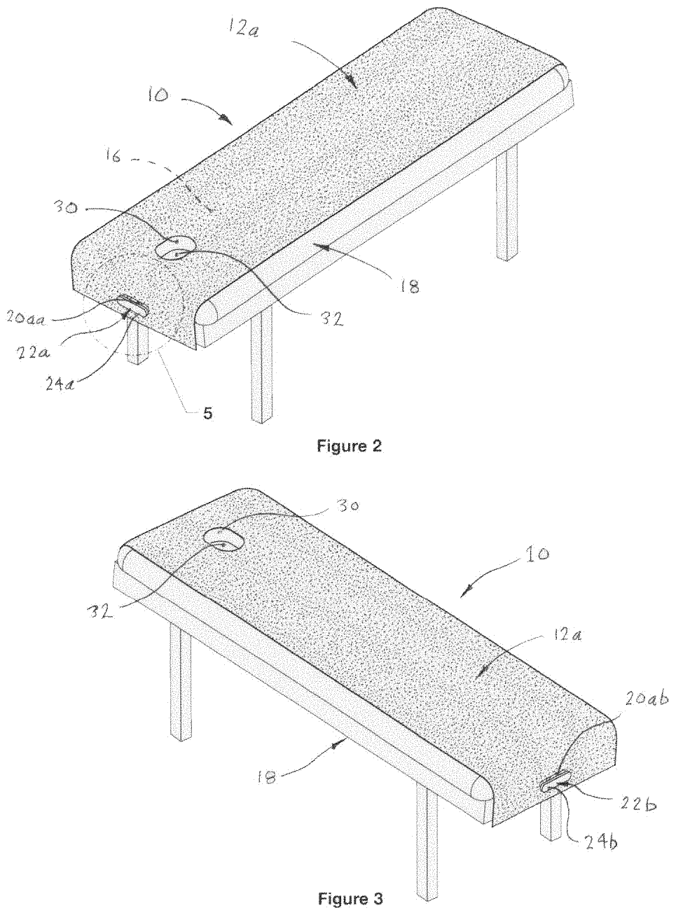

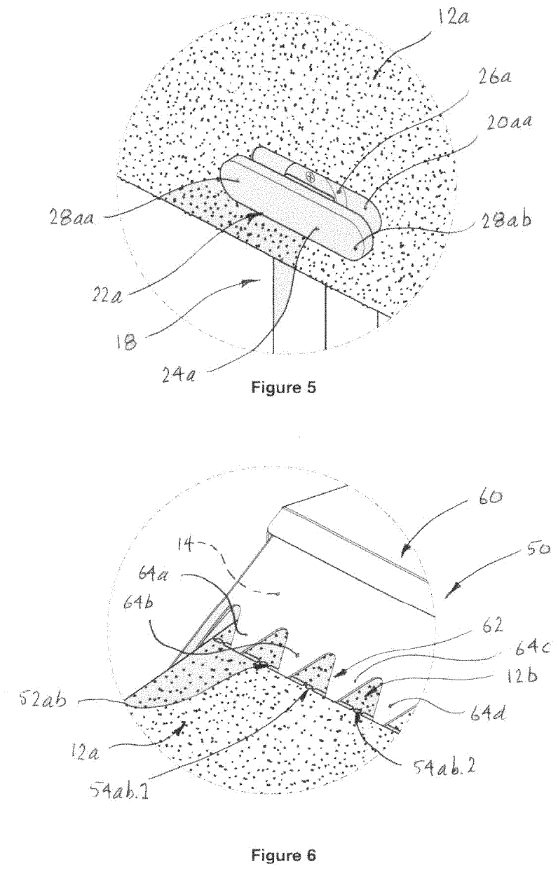

FIGS. 1 to 3 and 5 best illustrate the bed sheet and securement assembly 10 of an embodiment of the first aspect of the disclosure. The bed sheet and securement assembly 10 broadly includes:
1. the elongate sheet such as 12a being adapted to cover the exposed or shared upper surface 16 of the therapy bed 18, and including securement openings 20aa and 20ab formed adjacent to each of its opposing ends;
2. securement means 22a and 22b adapted to mount to the therapy bed 18 at or proximal its opposing ends and arranged for releasable engagement with the elongate sheet 12a via the securement openings 20aa and 20ab for securement of the elongate sheet 12a to the therapy bed 18.

The securement means such as 22a each include a hook fixture in the form of an elongate member such as 24a shaped at least in part complementary to the corresponding securement opening in the form of aperture 20aa. The aperture 20aa adjacent one end of the elongate sheet 12a is thus configured for releasable engagement with the corresponding elongate member 24a for securement of said sheet 12a to the therapy bed 18. Likewise, the elongate sheet 12a at an opposite end includes a securement opening in the form of slotted opening 20ab for releasable engagement with the corresponding elongate member 24b of the hook fixture 22b for securement of the sheet 12a to the bed 18.

In this embodiment the hook fixtures such as 24a and 24b include a base mounting 26a and 26b to which the corresponding elongate member 24a and 24b is connected. The base mounting such as 26a facilitates mounting of the hook fixture 22a to the therapy bed 18 where in this example it is screw fastened to the therapy bed 18. The elongate member such as 24a includes a pair of prongs 28aa and 28ab which extend laterally either side of the base mounting 26a. The prongs 28aa/ab are designed for releasable engagement of the elongate sheet 12a about its aperture 20aa for effective securement of the sheet 12a to the therapy bed 18. It is to be understood that the securement means in an alternative embodiment may be integrated with the bed 18 in the course of its manufacture rather than requiring fixing of the securement means 22a/b to the bed 18 as described in this embodiment. In either case the securement openings such as 20aa/ab are longitudinally separated a predetermined distance for fitting of the sheet 12a to the therapy bed 18 to prevent or limit the likelihood of the sheet 12a slipping on the bed 18. The securement openings 20aa/ab align with corresponding of the hook fixtures 22a/b wherein the elongate sheet completely covers the exposed or shared surface 16 of the therapy bed 18.

In this embodiment of both aspects of the disclosure the elongate sheet such as 12a is formed in one piece from a recyclable and non-woven material which is typically disposable. The recyclable material is in this example formed in multiple layers and is of a combined material density which is effective in bacterial filtration, for example the combined layers of the material has a density of around 28GSM. The material from which the sheet 12a of this embodiment is constructed is an SMMS non-woven material such as that published at www.sf-nonwoven.com/smms-nonwovenfabric/. It is to be understood that the sheet may be constructed of different combinations of non-woven layers.

The elongate sheet 12a of this embodiment includes a face hole opening 30 which aligns with a corresponding face hole opening 32 in the therapy bed 18. In this scenario the sheet 12a may cooperate with a face sheet (not shown) which fits about the face opening 30 and drapes within the face opening 32. This optional face sheet provides additional coverage for exposed surfaces surrounding the face opening 32 of the therapy bed 18 while a patient or client is lying prone or face down. The optional face sheet is constructed in accordance with the applicant's related technology disclosed in their Australian standard patent application no. 2017202118 and related applications. The disclosures and teachings of this Australian patent application are to be included herein by way of this reference. It is to be understood that, depending on the application to which it is applied, the elongate sheet may be formed with or without a face hole opening.

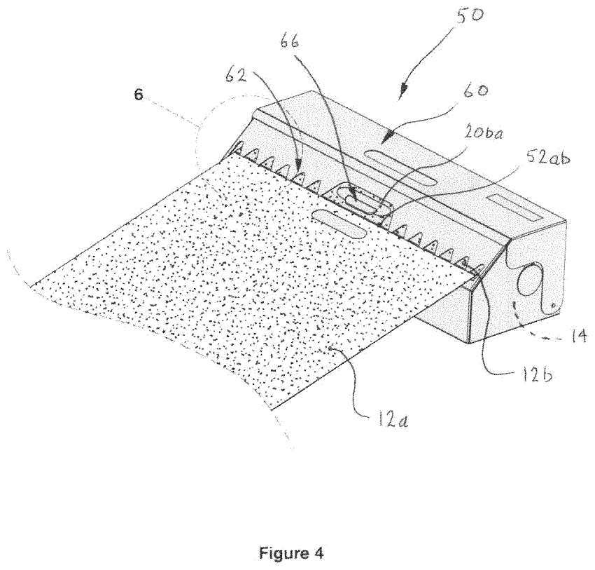

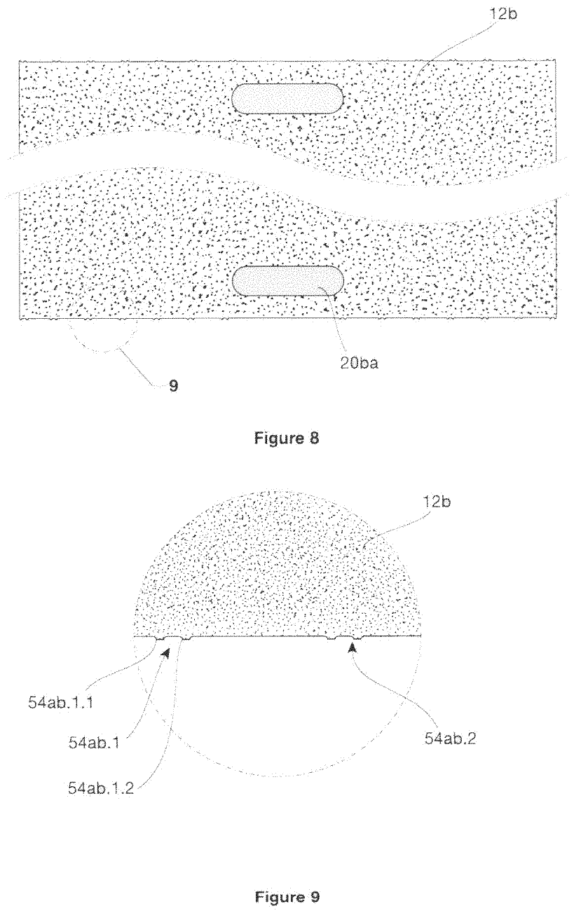

FIGS. 4 and 6 to 9 illustrate an embodiment of the bed sheet and dispenser assembly 50 according to the second aspect of the disclosure. The bed sheet and dispenser assembly 50 generally includes:
1. the dispensable roll 14 of the plurality of separate elongate sheets such as 12a and 12b each including:
   (a) securement openings such as 20aa and 20ab formed adjacent to each of its opposing ends and adapted for releasable engagement with securement means such as 22a and 22b mounted to the bed 18, and
   (b) a tear line such as 52ab defined by a plurality of tear junctions such 54ab.1 and 54ab.2 (see FIG. 9) for adjacent of the separable sheets 12a/b from the roll 14;
2. a sheet dispenser 60 configured for housing and tearing of the dispensable roll 14, said dispenser 60 including severing means depicted generally at 62 for alignment with the adjoining tear junctions such as 54ab.1 and 54ab.2 of adjacent of the sheets 12a/b for tearing of said sheets from the roll 14.

As seen in FIG. 6, the severing means 62 of this embodiment includes a series of teeth such as 64a to 64d arranged for engagement with the dispensable roll 14 across one of the tear lines such 52ab. The teeth such as 64c locate between neighbouring of the adjoining tear junctions such 54ab.1 and 54ab.2 to facilitate tearing of the sheets in this instance 12a from the adjacent sheet 12b of the roll 14. Each of the adjoining tear junctions such 54ab.1 includes a plurality of frangible tear points such as 56ab.1.1 and 54ab.1.2 (see FIG. 9). The assembly 50 is designed for manual withdrawal of the sheet such as 12a from the dispenser 60 by gripping of the associated aperture 20aa. The dispenser automatically separates the sheets 12a and 12b when the tear line 52ab aligns with the severing means 62 and continued manual withdrawal of the sheet 12a forces the teeth such as 64c into tearing engagement with neighbouring tear junctions 54ab.1 and 54ab.2 for tearing of said junctions.

As seen in FIGS. 1 and 4, the sheet dispenser 60 includes capture means 66 in this example aligned with the severing means 62 and configured for releasable engagement of one of the securement openings such as 20ba. It is to be understood that this releasable engagement of the securement opening or aperture 20ba restricts retraction of the associated sheet 12b within the dispenser 60 during dispensing of the sheet 12a from the roll 14. In this example the capture means is in the form of a fixed latching member 66 which is configured to cooperate with the apertures such as 20ba. In the event the sheet 12b is latched by the latching member 66, the associated sheet 12b can with relative ease be extracted from the dispenser 60 by grabbing the aperture such as 20ba and drawing the associated sheet 12b outward of the dispenser 60 whereupon it is released from the latching member 66.

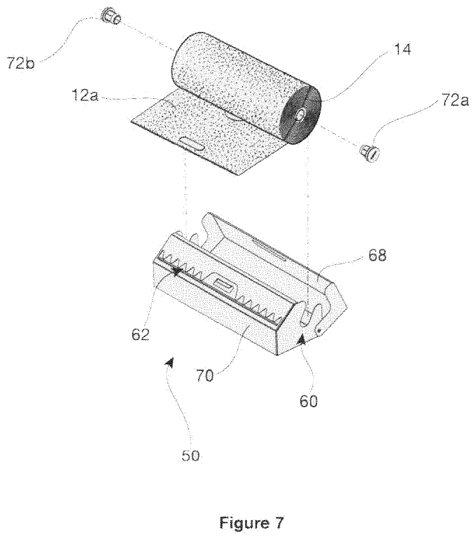

FIG. 7 schematically illustrates loading of the dispensable roll 14 of the separable elongate sheets such as 12a into the sheet dispenser 60. The sheet dispenser 60 includes a hinged lid 68 which is folded outward of an associated housing 70 in its open configuration. In this open configuration, the roll 14 is lowered inside the housing 70 and retained for rolling within the housing 70 via retractable roller plugs 72a and 72b. The outermost of the sheets 12a is fed underneath the severing means 62 in readiness for dispensing of elongate sheets such as 12a. The hinged lid 68 is then moved into its closed configuration to enclose the roll 14 as seen for example in FIGS. 1 and 4.

Now that an embodiment of the alternative aspects of the disclosure have been described it will be apparent to those skilled in the art that they have at least the following advantages:
1. the elongate sheet is configured for releasable engagement with securement means associated with a therapy or other bed for quick and convenient coverage of the bed and its exposed and/or shared surfaces;
2. the elongate sheet includes securement openings which cooperate with securement means mounted to the bed without relying upon additional fastenings or clips;
3. the bed sheet and securement assembly provides effective fitting to the therapy bed without incorporating elastic, mixed materials or other non-disposable or non-recyclable means incorporated within the fitted sheet;
4. the securement means provide relatively simple and functionally effective means for anchoring the elongate sheet without requiring supplementary fastenings or clip arrangements;
5. the bed sheet and dispenser assembly provides relatively quick and effective means for dispensing and separating elongate sheets at predetermined lengths;
6. the dispensable sheets include securement openings which are of a dual function providing securement of the sheet to an associated bed and if necessary cooperation with the dispenser to prevent retraction of the roll during dispensing.

Those skilled in the art will appreciate that the embodiments described herein are susceptible to variations and modifications other than those specifically described. For example, the securement openings and associated securement means may vary in their shape and construction provided they permit releasable inter-engagement which is effective in securement of the sheet to the bed. The securement openings and securement means may vary in number where for example the securement means take the form of rigid buttons located at or proximal opposing ends of the bed and arranged to cooperate with securement openings such as slits in each of the corners of the elongate sheet. Depending on the type and size of bed, the securement means may be mounted to the bed underneath its opposing ends. The form of the severing means of the sheet dispenser may vary depending of the construction of the tear line in adjoining sheets of the roll. The dispenser assembly may be rigidly mounted to a floor or wall adjacent the bed or, depending on the weight of the assembly, merely placed on the floor without any anchorage. Alternatively the dispenser assembly may be associated with or mounted to the bed itself.

All such variations and modifications are to be considered within the scope of the present disclosure the nature of which is to be determined from the foregoing description.

What is claimed is:

1. A bed sheet and securement assembly comprising:
an elongate sheet being one of a plurality of sheets provided in a dispensable roll of separable sheets, said elongate sheet being adapted to cover exposed or shared surfaces of a bed, and including securement openings formed adjacent to each of opposing ends of said sheet; and
securement means adapted to mount to the bed at or proximal its opposing ends and arranged for releasable engagement with the elongate sheet via the securement openings for securement of the elongate sheet to the bed.

2. A bed sheet and securement assembly as claimed in claim 1, wherein the securement means each include a hook fixture configured to cooperate with respective of the securement openings formed in the elongate sheet for releasable engagement of said openings with said hook fixtures for securement of said sheet to the bed.

3. A bed sheet and securement assembly as claimed in claim 2, wherein each of the hook fixtures includes an elongate member connected to a base mounting of the securement for mounting to the bed, the securement openings being shaped at least in part complementary to the elongate member.

4. A bed sheet and securement assembly as claimed in claim 3, wherein the elongate member includes a pair of prongs extending laterally either side of the base mounting and configured for releasable engagement of the elongate sheet about its securement openings.

5. A bed sheet and securement assembly as claimed in claim 2, wherein the securement openings of the elongate sheet are each formed as an aperture in said sheet.

6. A bed sheet and securement assembly claimed in claim 5, wherein the apertures are aligned with the elongate member of the hook members mounted at or proximal each end of the bed.

7. A bed sheet and securement assembly as claimed in claim 6, wherein the apertures are longitudinally separated a predetermined distance for fitting of the elongate sheet to the exposed or shared surface of the bed via the hook fixtures mounted at opposing ends of said bed.

8. A bed sheet and securement assembly as claimed in claim 1, wherein the elongate sheet is formed in one piece from a recyclable and non-woven material.

9. A bed sheet and securement assembly as claimed in claim 8, wherein the recyclable material is formed in multiple layers being effective in bacterial filtration.

10. A bed sheet and securement assembly as claimed in claim 8, wherein the one-piece elongate sheet completely covers the exposed or shared surface of the bed.

11. A bed sheet and securement assembly as claimed in claim 9, wherein the one-piece elongate sheet completely covers the exposed or shared surface of the bed.

12. A bed sheet and dispenser assembly comprising:
a dispensable roll of a plurality of separable elongate sheets each being adapted to cover an exposed or shared surface of a bed, said sheets each including securement openings formed adjacent to each of opposing ends of said sheet and adapted for releasable engagement with securement means mounted to the bed for securement of said sheet to said bed, and a tear line defined by a plurality of adjoining tear junctions for adjacent of the separable sheets from the roll; and
a sheet dispenser configured for housing and tearing of the dispensable roll, said dispenser including severing means arranged for alignment with the adjoining tear junctions of adjacent of the sheets for tearing of each of said sheets from the roll.

13. A bed sheet and dispenser assembly as claimed in claim 12, wherein the severing means includes a series of teeth arranged for engagement with the dispensable roll across one of the tear lines wherein the teeth locate between neighbouring of the adjoining tear junctions to facilitate tearing of each of said sheets from the roll.

14. A bed sheet and dispenser assembly as claimed in claim 13, wherein each of the adjoining tear junctions includes a plurality of frangible tear points located in close proximity to one another.

15. A bed sheet and dispenser assembly as claimed in claim 12, wherein the sheet dispenser includes capture means located adjacent the severing means and configured for releasable engagement of one of the securement openings to restrict retraction of the associated sheet within the dispenser during dispensing of one of the sheets from the roll.

16. A bed sheet and dispenser assembly as claimed in claim 15, wherein the capture means includes a fixed latching member configured to cooperate with the securement opening to latch with said opening in the event the associated sheet retracts within the dispenser.
